(19) United States
(12) Patent Application Publication
(10) Pub. No.: US 2008/0280759 A1
(43) Pub. Date: Nov. 13, 2008

(54) 6-(2-Fluoro-4-Alkoxyphenyl) Triazolopyrimidines, Their Preparation, Their Use For Controlling Harmful Fungi, and Compositions Comprising Them

(76) Inventors: Jordi Tormo i Blasco, Laudenbach (DE); Carsten Blettner, Mannheim (DE); Bernd Muller, Frankenthal (DE); Markus Gewehr, Kastellaun (DE); Wassilios Grammenos, Ludwigshafen (DE); Thomas Grote, Wachenheim (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Maria Scherer, Godramstein (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 10/582,971

(22) PCT Filed: Dec. 14, 2004
(86) PCT No.: PCT/EP04/14228

§ 371 Date: Jun. 15, 2006

(30) Foreign Application Priority Data
Dec. 17, 2003 (DE) 103 59 435.3

(51) Int. Cl. A01C 1/06; C07D 487/04; A61K 31/519

(52) U.S. Cl. 504/100; 544/263; 514/259.31

(57) ABSTRACT

6-(2-Halo-4-alkoxyphenyl)triazolopyrimidines of the formula I

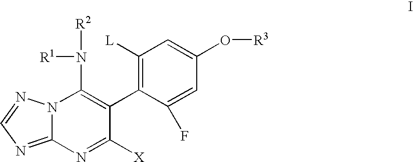

in which the substituents are as defined below:
R¹ is alkyl, haloalkyl, cycloalkyl, halocycloalkyl, alkenyl, haloalkenyl, cycloalkenyl, halocycloalkenyl, alkynyl, haloalkynyl or phenyl, naphthyl, or a five- or six-membered saturated, partially unsaturated or aromatic heterocycle which contains one to four heteroatoms from the group consisting of O, N and S,
R² is hydrogen or one of the groups mentioned under R¹,
   R¹ and R² together with the nitrogen atom to which they are attached may also form a five- or six-membered heterocyclyl or heteroaryl which is attached via N and may contain one to three further heteroatoms from the group consisting of O, N and S as ring members;
R³ is alkyl, haloalkyl, alkenyl, haloalkenyl, alkynyl, haloalkynyl, phenylalkyl, mono- or dialkoxyalkyl;
   R¹, R² and/or R³ may be substituted as mentioned in the description;
L is hydrogen, fluorine or chlorine;
X is cyano, alkyl, alkoxy, alkenyloxy, haloalkoxy or haloalkenyloxy;
   processes for preparing these compounds, compositions comprising them and their use for controlling phytopathogenic harmful fungi.

The present invention relates to 6-(2-halo-4-alkoxyphenyl)triazolopyrimidines of the formula I

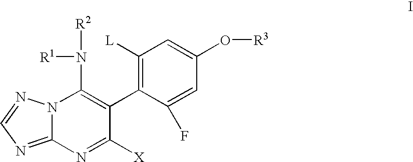

in which the substituents are as defined below:

R¹ C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which contains one to four heteroatoms from the group consisting of O, N and S,

R² is hydrogen or one of the groups mentioned under R¹,
R¹ and R² together with the nitrogen atom to which they are attached may also form a five- or six-membered heterocyclyl or heteroaryl which is attached via N and may contain one to three further heteroatoms from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;
R³ is C₁-C₈-alkyl, C₁-C₈-haloalkyl, C₃-C₈-alkenyl, C₃-C₈-haloalkenyl, C₃-C₈-alkynyl, C₃-C₈-haloalkynyl, phenyl, phenyl-C₁-C₄-alkyl, mono- or di-(C₁-C₄-alkoxy)-C₁-C₄-alkyl;

R¹, R² and/or R³ may carry one to four identical or different groups R^{a}:

R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₂-C₈-haloalkenyl, C₂-C₆-alkenyloxy, C₂-C₈-alkynyl, C₂-C₈-haloalkynyl, C₃-C₆-alkynyloxy, oxy-C₁-C₃-alkyleneoxy, C₃-C₈-cycloalkenyl, phenyl, naphthyl, a five- or six-membered saturated, partially unsaturated or aromatic heterocycle which contains one to four heteroatoms from the group consisting of O, N and S, where these aliphatic, alicyclic or aromatic groups for their part may be partially or fully halogenated;

L is hydrogen, fluorine or chlorine; and

X is cyano, C₁-C₄-alkyl, C₁-C₄-alkoxy, C₃-C₄-alkenyloxy, C₁-C₂-haloalkoxy or C₃-C₄-haloalkenyloxy.

Moreover, the invention relates to a process for preparing these compounds, to compositions comprising them and to their use for controlling phytopathogenic harmful fungi.

5-Halo-6-(2-halo-4-alkoxyphenyl)triazolopyrimidines are known in a general manner from WO 99/48893. 5-Cyano- and 5-alkoxytriazolopyrimidines are disclosed in WO 02/083677. Triazolopyrimidines having optically active amino substituents in the 7-position are proposed in a general manner in WO 02/38565.

The compounds described in the publications mentioned above are suitable for controlling harmful fungi.

However, their action is not always entirely satisfactory. It is therefore an object of the present invention to provide compounds having improved activity and/or a broader activity spectrum.

We have found that this object is achieved by the compounds defined at the outset. Moreover, we have found a process for their preparation, compositions comprising them and methods for controlling harmful fungi using the compounds I.

The compounds according to the invention differ from the compounds described in the publications above by the substitution in the 5-position of the triazolopyrimidine skeleton.

Compared to the known compounds, the compounds of the formula I have increased activity and/or a broader activity spectrum against harmful fungi.

The compounds according to the invention can be obtained by different routes. Advantageously, they are obtained starting with the 5-halo-6-(2-halo4-alkoxyphenyl)triazolopyrimidines of the formula II known from WO 99/48893, by reaction with compounds M-X (formula III). Depending on the meaning of the group X to be introduced, the compounds III are inorganic cyanides or alkoxylates. The reaction is advantageously carried out in the presence of an inert solvent. The meaning of the cation M in formula III is of little importance; for practical reasons, ammonium, tetraalkylammonium or alkali metal or alkaline earth metal salts are usually preferred.

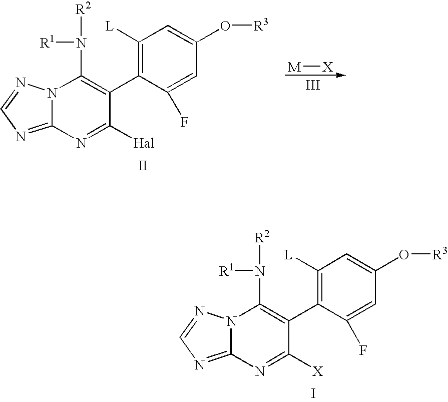

The reaction temperature is usually from 0 to 120° C., preferably from 10 to 40° C. [cf. J. Heterocycl. Chem., 12 (1975), S. 861-863].

If R² is hydrogen, a removable protective group is advantageously introduced prior to the reaction with III [cf. Greene, Protective Groups in Organic Chemistry, J. Wiley & Sons, (1981)].

Suitable solvents include ethers, such as dioxane, diethyl ether and, preferably, tetrahydrofuran, alcohols, such as methanol or ethanol, halogenated hydrocarbons, such as dichloromethane, and aromatic hydrocarbons, such as toluene or acetonitrile.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

Compounds of the formula I in which X is C₁-C₄-alkyl can be obtained advantageously by the synthesis route below:

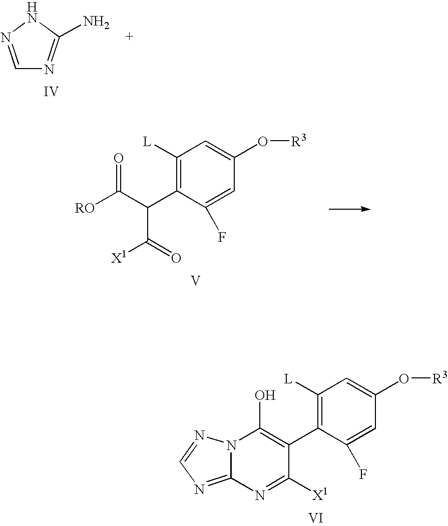

The 5-alkyl-7-hydroxy-6-phenyltriazolopyrimidines VI are obtained from 2-aminotriazole IV and keto esters V. In the formulae V and VI, X¹ is C₁-C₄-alkyl. Using the easily obtainable 2-phenylacetoacetic esters (V where X¹=CH₃), the 5-methyl-7-hydroxy-6-phenyltriazolopyrimidines, which are a preferred subject-matter of the invention, are obtained [cf. Chem. Pharm. Bull., 9, 801 (1961)]. 2-Aminotriazole IV is commercially available. The starting materials V are advantageously prepared under the conditions described in EP-A 10 02 788.

The 5-alkyl-7-hydroxy-6-phenyltriazolopyrimidines obtained in this manner are, using halogenating agents [HAL], converted under the conditions known from WO-A 94/20501 into the halopyrimidines of the formula VII in which Hal is a halogen atom, preferably a bromine or a chlorine atom, in particular a chlorine atom. Advantageous halogenating agents [HAL] are chlorinating or brominating agents, such as phosphorus oxybromide, phosphorus oxychloride, thionyl chloride, thionyl bromide or sulfuryl chloride.

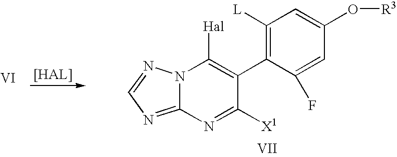

This reaction is usually carried out at from 0° C. to 150° C., preferably at from 80° C. to 125° C. [cf. EP-A 770 615].

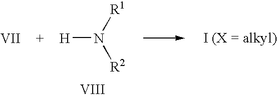

The reaction of VII with amines VIII in which R¹ and R² are as defined for formula I is advantageously carried out at from 0° C. to 70° C., preferably from 10° C. to 35° C., preferably in the presence of an inert solvent, such as an ether, for example dioxane, diethyl ether or, in particular, tetrahydrofuran, a halogenated hydrocarbon, such as dichloromethane, or an aromatic hydrocarbon, such as, for example, toluene [cf. WO-A 98/46608].

Preference is given to using a base, such as a tertiary amine, for example triethylamine, or an inorganic amine, such as potassium carbonate; it is also possible for excess amine of the formula VIII to serve as base.

Alternatively, compounds of the formula I in which X is C₁-C₄-alkyl can also be prepared from compounds I in which X is halogen, in particular chlorine, and malonates of the formula IX. In formula IX, X″ is hydrogen or C₁-C₃-alkyl and R is C₁-C₄-alkyl. They are converted into compounds of the formula X and decarboxylated to compounds I [cf. U.S. Pat. No. 5,994,360].

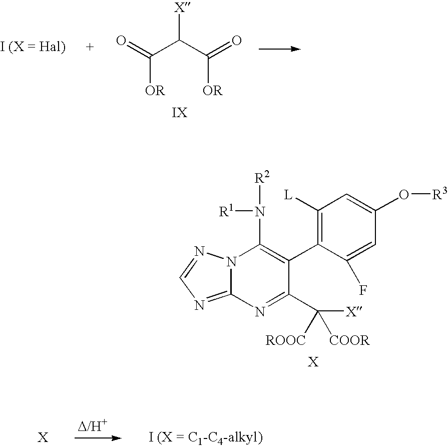

The malonates IX are known from the literature [J. Am. Chem. Soc. 64 (1942), 2714; J. Org. Chem. 39 (1974), 2172; Helv. Chim. Acta 61 (1978), 1565], or they can be prepared in accordance with the literature cited.

The subsequent hydrolysis of the ester X is carried out under generally customary conditions; depending on the different structural elements, alkaline or acidic hydrolysis of compounds X may be advantageous. Under the conditions of ester hydrolysis, there may already be complete or partial decarboxylation to I.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preference, the reaction is carried out in hydrochloric acid or acetic acid. It is also possible to use mixtures of the solvents mentioned.

Compounds of the formula I in which X is C₁-C₄-alkyl can also be obtained by coupling 5-halotriazolopyrimidines of the formula I in which X is halogen with organometallic reagents of the formula XI. In one embodiment of this process, the reaction is carried out with transition metal catalysis, such as Ni or Pd catalysis.

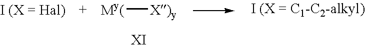

In formula XI, M is a metal ion of valency Y, such as, for example, B, Zn or Sn, and X″ is C₁-C₃-alkyl. This reaction can be carried out, for example, analogously to the following methods: J. Chem. Soc. Perkin Trans. 1 (1994), 1187, ibid. 1 (1996), 2345; WO-A 99/41255; Aust. J. Chem. 43 (1990), 733; J. Org. Chem. 43 (1978), 358; J. Chem. Soc. Chem. Commun. (1979), 866; Tetrahedron Lett. 34 (1993), 8267; ibid. 33 (1992), 413.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

In the definitions of the symbols given in the formulae above, collective terms were used which are generally representative of the following substituents:

halogen: fluorine, chlorine, bromine and iodine;

alkyl: saturated straight-chain or branched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl,1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4, 6 or 8 carbon atoms (as mentioned above), where in these groups some or all of the hydrogen atoms may be replaced by halogen atoms as mentioned above; in particular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated straight-chain or branched hydrocarbon radicals having 2 to 8 carbon atoms and one or two double bonds in any position (as mentioned above), where in these groups some or all of the hydrogen atoms may be replaced by halogen atoms as mentioned above, in particular by fluorine, chlorine and bromine;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;

five- or six-membered saturated, partially unsaturated or aromatic heterocycle which contains one to four heteroatoms from the group consisting of O, N and S:

5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolid inyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;

5-membered heteroaryl which contains one to four nitrogen atoms or one to three nitrogen atoms and one sulfur or oxygen atom: 5-Ring heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;

6-membered heteroaryl which contains one to three or one to four nitrogen atoms: 6-Ring heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;

alkylene: saturated, straight-chain or branched hydrocarbon radicals having 1 to 4 or 6 carbon atoms, which radicals are attached to the skeleton via a double bond, for example ═CH₂, ═CH—CH₃, ═CH—CH₂—CH₃; oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O.

The scope of the present invention includes the (R)- and (S)-isomers and the racemates of compounds of the formula I having chiral centers.

With a view to the intended use of the triazolopyrimidines of the formula I, particular preference is given to the following meanings of the substituents, in each case on their own or in combination:

Preference is given to compounds I in which R¹ is C₁-C₄-alkyl, C₂-C₆-alkenyl or C₁-C₈-haloalkyl.

Preference is given to compounds I in which R¹ is a group A:

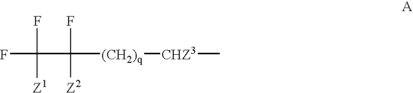

in which

Z¹ is hydrogen, fluorine or C₁-C₆-fluoroalkyl,

Z² is hydrogen or fluorine, or
Z¹ and Z² together form a double bond;

q is 0 or 1; and

Z³ is hydrogen or methyl.

Moreover, preference is given to compounds I in which R¹ is C₃-C₆-cycloalkyl which may be substituted by C₁-C₄-alkyl.

Especially preferred are compounds I in which R² is hydrogen.

Preference is likewise given to compounds I in which R² is methyl or ethyl.

If R¹ and/or R² comprise haloalkyl or haloalkenyl groups having a center of chirality, the (S)-isomers are preferred for these groups. In the case of halogen-free alkyl or alkenyl groups having a center of chirality in R¹ or R², preference is given to the (R)-configured isomers.

A preferred embodiment of the invention relates to compounds of the formula I.1:

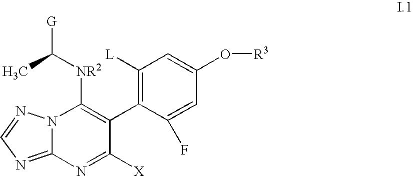

in which

G is C₂-C₆-alkyl, in particular ethyl, n- and isopropyl, n-, sec-, tert-butyl, and C₁-C₄-alkoxymethyl, in particular ethoxymethyl, or C₃-C₆-cycloalkyl, in particular cyclopentyl or cyclohexyl;

R² is hydrogen or methyl;

X, L and R³ are as defined at the outset, where X is, in particular, cyano, methoxy or ethoxy.

A further preferred embodiment of the invention relates to compounds of the formula I.2.

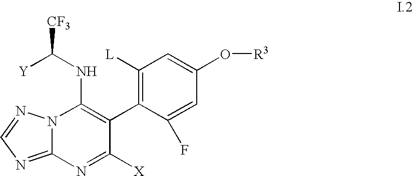

in which Y is hydrogen or C₁-C₄-alkyl, in particular methyl and ethyl, and X, L and R³ are as defined at the outset, where X is, in particular, cyano, methoxy or ethoxy.

A further preferred embodiment of the invention relates to compounds in which R¹ and R² together with the nitrogen atom to which they are attached form a five- or six-membered heterocyclyl or heteroaryl which is attached via N and may contain a further heteroatom from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₁-C₆-alkylene and oxy-C₁-C₃-alkyleneoxy. These compounds correspond in particular to formula I.3,

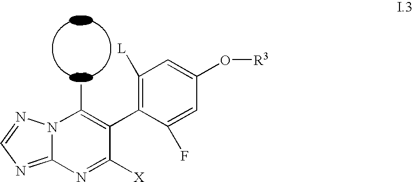

in which

D together with the nitrogen atom forms a five- or six-membered heterocyclyl or heteroaryl which is attached via N and may contain a further heteroatom from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;

X, L and R³ are as defined at the outset, where X is, in particular, cyano, methoxy or ethoxy.

Particular preference is given to compounds of the formula I.3, in which L is hydrogen and R³ is methyl.

Preference is furthermore given to compounds I in which R¹ and R² together with the nitrogen atom to which they are attached form a piperidinyl, morpholinyl or thiomorpholinyl ring, in particular a piperidinyl ring, which, if appropriate, is substituted by one to three groups of halogen, C₁-C₄-alkyl or C₁-C₄-haloalkyl. Particular preference is given to the compounds in which R¹ and R² together with the nitrogen atom to which they are attached form a 4-methylpiperidine ring.

The invention furthermore preferably provides compounds I in which R¹ and R² together with the nitrogen atom to which they are attached form a pyrazole ring which, if appropriate, is substituted by one or two groups of halogen, C₁-C₄-alkyl or C₁-C₄-haloalkyl, in particular by 3,5-dimethyl or 3,5-di(trifluoromethyl).

The invention particularly preferably provides compounds of the formula I in which X is cyano, methoxy or ethoxy, in particular cyano or methoxy.

In another preferred embodiment of the compounds of the formula I, X is C₁-C₄-alkyl, in particular methyl.

In addition, particular preference is also given to compounds of the formula I in which R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl; R² is hydrogen or methyl; or R¹ and R² together are —(CH₂)₂CH(CH₃)(CH₂)₂-, —(CH₂)₂CH(CF₃)(CH₂)₂— or —(CH₂)₂O (CH₂)₂-, in particular to those in which X is cyano or methoxy.

The invention furthermore preferably provides compounds of the formula I in which R³ is alkyl, in particular methyl.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

### Table 1

Compounds of the formula I, in which L is hydrogen, R³ is methyl and X is cyano and the combination of R¹ and R² corresponds for each compound to one row of Table A

### Table 2

Compounds of the formula I, in which L is fluorine, R³ is methyl and X is cyano and the combination of R¹ and R² corresponds for each compound to one row of Table A

### Table 3

Compounds of the formula I, in which L is chlorine, R³ is methyl and X is cyano and the combination of R¹ and R² corresponds for each compound to one row of Table A

### Table 4

Compounds of the formula I, in which L is hydrogen, R³ is ethyl and X is cyano and the combination of R¹ and R² corresponds for each compound to one row of Table A

### Table 5

Compounds of the formula I, in which L is fluorine, R³ is ethyl and X is cyano and the combination of R¹ and R² corresponds for each compound to one row of Table A

### Table 6

Compounds of the formula I, in which L is chlorine, R³ is ethyl and X is cyano and the combination of R¹ and R² corresponds for each compound to one row of Table A

### Table 7

Compounds of the formula I, in which L is hydrogen, R³ is n-propyl and X is cyano and the combination of R¹ and R² corresponds for each compound to one row of Table A

### Table 8

Compounds of the formula I, in which L is fluorine, R³ is n-propyl and X is cyano and the combination of R¹ and R² corresponds for each compound to one row of Table A

### Table 9

Compounds of the formula I, in which L is chlorine, R³ is n-propyl and X is cyano and the combination of R¹ and R² corresponds for each compound to one row of Table A

### Table 10

Compounds of the formula I, in which L is hydrogen, R³ is isopropyl and X is cyano and the combination of R¹ and R² corresponds for each compound to one row of Table A

### Table 11

Compounds of the formula I, in which L is fluorine, R³ is isopropyl and X is cyano and the combination of R¹ and R² corresponds for each compound to one row of Table A

### Table 12

Compounds of the formula I, in which L is chlorine, R³ is isopropyl and X is cyano and the combination of R¹ and R² corresponds for each compound to one row of Table A

### Table 13

Compounds of the formula I, in which L is hydrogen, R³ is 2-fluoroethyl and X is cyano and the combination of R¹ and R² corresponds for each compound to one row of Table A

### Table 14

Compounds of the formula I, in which L is fluorine, R³ is 2-fluoroethyl and X is cyano and the combination of R¹ and R² corresponds for each compound to one row of Table A

### Table 15

Compounds of the formula I, in which L is chlorine, R³ is 2-fluoroethyl and X is cyano and the combination of R¹ and R² corresponds for each compound to one row of Table A

### Table 16

Compounds of the formula I, in which L is hydrogen, R³ is allyl and X is cyano and the combination of R¹ and R² corresponds for each compound to one row of Table A

### Table 17

Compounds of the formula I, in which L is fluorine, R³ is allyl and X is cyano and the combination of R¹ and R² corresponds for each compound to one row of Table A

### Table 18

Compounds of the formula I, in which L is chlorine, R³ is allyl and X is cyano and the combination of R¹ and R² corresponds for each compound to one row of Table A

### Table 19

Compounds of the formula I, in which L is hydrogen, R³ is 2-methoxyethyl and X is cyano and the combination of R¹ and R² corresponds for each compound to one row of Table A

### Table 20

Compounds of the formula I, in which L is fluorine, R³ is 2-methoxyethyl and X is cyano and the combination of R¹ and R² corresponds for each compound to one row of Table A

### Table 21

Compounds of the formula I, in which L is chlorine, R³ is 2-methoxyethyl and X is cyano and the combination of R¹ and R² corresponds for each compound to one row of Table A

### Table 22

Compounds of the formula I, in which L is hydrogen, R³ is methyl and X is methoxy and the combination of R¹ and R² corresponds for each compound to one row of Table A

### Table 23

Compounds of the formula I, in which L is fluorine, R³ is methyl and X is methoxy and the combination of R¹ and R² corresponds for each compound to one row of Table A

### Table 24

Compounds of the formula I, in which L is chlorine, R³ is methyl and X is methoxy and the combination of R¹ and R² corresponds for each compound to one row of Table A

### Table 25

Compounds of the formula I, in which L is hydrogen, R³ is ethyl and X is methoxy and the combination of R¹ and R² corresponds for each compound to one row of Table A

### Table 26

Compounds of the formula I, in which L is fluorine, R³ is ethyl and X is methoxy and the combination of R¹ and R² corresponds for each compound to one row of Table A

### Table 27

Compounds of the formula I, in which L¹ is chlorine, R³ is ethyl and X is methoxy and the combination of R¹ and R² corresponds for each compound to one row of Table A

### Table 28

Compounds of the formula I, in which L is hydrogen, R³ is n-propyl and X is methoxy and the combination of R¹ and R² corresponds for each compound to one row of Table A

### Table 29

Compounds of the formula I, in which L is fluorine, R³ is n-propyl and X is methoxy and the combination of R¹ and R² corresponds for each compound to one row of Table A

### Table 30

Compounds of the formula I, in which L is chlorine, R³ is n-propyl and X is methoxy and the combination of R¹ and R² corresponds for each compound to one row of Table A

### Table 31

Compounds of the formula I, in which L is hydrogen, R³ is isopropyl and X is methoxy and the combination of R¹ and R² corresponds for each compound to one row of Table A

### Table 32

Compounds of the formula I, in which L is fluorine, R³ is isopropyl and X is methoxy and the combination of R¹ and R² corresponds for each compound to one row of Table A

### Table 33

Compounds of the formula I, in which L is chlorine, R³ is isopropyl and X is methoxy and the combination of R¹ and R² corresponds for each compound to one row of Table A

### Table 34

Compounds of the formula I, in which L is hydrogen, R³ 2-fluoroethyl and X is methoxy and the combination of R¹ and R² corresponds for each compound to one row of Table A

### Table 35

Compounds of the formula I, in which L is fluorine, R³ is 2-fluoroethyl and X is methoxy and the combination of R¹ and R² corresponds for each compound to one row of Table A

### Table 36

Compounds of the formula I, in which L is chlorine, R³ is 2-fluoroethyl and X is methoxy and the combination of R¹ and R² corresponds for each compound to one row of Table A

### Table 37

Compounds of the formula I, in which L is hydrogen, R³ is allyl and X is methoxy and the combination of R¹ and R² corresponds for each compound to one row of Table A

### Table 38

Compounds of the formula I, in which L is fluorine, R³ is allyl and X is methoxy and the combination of R¹ and R² corresponds for each compound to one row of Table A

### Table 39

Compounds of the formula I, in which L is chlorine, R³ is allyl and X is methoxy and the combination of R¹ and R² corresponds for each compound to one row of Table A

### Table 40

Compounds of the formula I, in which L is hydrogen, R³ is 2-methoxyethyl and X is methoxy and the combination of R¹ and R² corresponds for each compound to one row of Table A

### Table 41

Compounds of the formula I, in which L is fluorine, R³ is 2-methoxyefhyl and X is methoxy and the combination of R¹ and R² corresponds for each compound to one row of Table A

### Table 42

Compounds of the formula I, in which L is chlorine, R³ is 2-methoxyethyl and X is methoxy and the combination of R¹ and R² corresponds for each compound to one row of Table A

### Table 43

Compounds of the formula I, in which L is hydrogen, R³ is methyl and X is ethoxy and the combination of R¹ and R² corresponds for each compound to one row of Table A

### Table 44

Compounds of the formula I, in which L is fluorine, R³ is methyl and X is ethoxy and the combination of R¹ and R² corresponds for each compound to one row of Table A

### Table 45

Compounds of the formula I, in which L is chlorine, R³ is methyl and X is ethoxy and the combination of R¹ and R² corresponds for each compound to one row of Table A

### Table 46

Compounds of the formula I, in which L is hydrogen, R³ is ethyl and X is ethoxy and the combination of R¹ and R² corresponds for each compound to one row of Table A

### Table 47

Compounds of the formula I, in which L is fluorine, R³ is ethyl and X is ethoxy and the combination of R¹ and R² corresponds for each compound to one row of Table A

### Table 48

Compounds of the formula I, in which L is chlorine, R³ is ethyl and X is ethoxy and the combination of R¹ and R² corresponds for each compound to one row of Table A

### Table 49

Compounds of the formula I, in which L is hydrogen, R³ is n-propyl and X is ethoxy and the combination of R¹ and R² corresponds for each compound to one row of Table A

### Table 50

Compounds of the formula I, in which L is fluorine, R³ is n-propyl and X is ethoxy and the combination of R¹ and R² corresponds for each compound to one row of Table A

### Table 51

Compounds of the formula I, in which L is chlorine, R³ is n-propyl and X is ethoxy and the combination of R¹ and R² corresponds for each compound to one row of Table A

### Table 52

Compounds of the formula I, in which L is hydrogen, R³ is isopropyl and X is ethoxy and the combination of R¹ and R² corresponds for each compound to one row of Table A

### Table 53

Compounds of the formula I, in which L is fluorine, R³ is isopropyl and X is ethoxy and the combination of R¹ and R² corresponds for each compound to one row of Table A

### Table 54

Compounds of the formula I, in which L is chlorine, R³ is isopropyl and X is ethoxy and the combination of R¹ and R² corresponds for each compound to one row of Table A

### Table 55

Compounds of the formula I, in which L is hydrogen, R³ is 2-fluoroethyl and X is ethoxy and the combination of R¹ and R² corresponds for each compound to one row of Table A

### Table 56

Compounds of the formula I, in which L is fluorine, R³ is 2-fluoroethyl and X is ethoxy and the combination of R¹ and R² corresponds for each compound to one row of Table A

### Table 57

Compounds of the formula I, in which L is chlorine, R³ is 2-fluoroethyl and X is ethoxy and the combination of R¹ and R² corresponds for each compound to one row of Table A

### Table 58

Compounds of the formula I, in which L is hydrogen, R³ is allyl and X is ethoxy and the combination of R¹ and R² corresponds for each compound to one row of Table A

### Table 59

Compounds of the formula I, in which L is fluorine, R³ is allyl and X is ethoxy and the combination of R¹ and R² corresponds for each compound to one row of Table A

### Table 60

Compounds of the formula i, in which L is chlorine, R³ is allyl and X is ethoxy and the combination of R¹ and R² corresponds for each compound to one row of Table A

### Table 61

Compounds of the formula I, in which L is hydrogen, R³ is 2-methoxyethyl and X is ethoxy and the combination of R¹ and R² corresponds for each compound to one row of Table A

### Table 62

Compounds of the formula I, in which L is fluorine, R³ is 2-methoxyethyl and X is ethoxy and the combination of R¹ and R² corresponds for each compound to one row of Table A

### Table 63

Compounds of the formula I, in which L is chlorine, R³ is 2-methoxyethyl and X is ethoxy and the combination of R¹ and R² corresponds for each compound to one row of Table A

### Table 64

Compounds of the formula I, in which L is hydrogen, R³ is methyl and X is methyl and the combination of R¹ and R² corresponds for each compound to one row of Table A

### Table 65

Compounds of the formula I, in which L is fluorine, R³ is methyl and X is methyl and the combination of R¹ and R² corresponds for each compound to one row of Table A

### Table 66

Compounds of the formula I, in which L is chlorine, R³ is methyl and X is methyl and the combination of R¹ and R² corresponds for each compound to one row of Table A

### Table 67

Compounds of the formula I, in which L is hydrogen, R³ is ethyl and X is methyl and the combination of R¹ and R² corresponds for each compound to one row of Table A

### Table 68

Compounds of the formula I, in which L is flourine, R³ is ethyl and X is methyl and the combination of R¹ and R² corresponds for each compound to one row of Table A

### Table 69

Compounds of the formula I, in which L is chlorine, R³ is ethyl and X is methyl and the combination of R¹ and R² corresponds for each compound to one row of Table A

### Table 70

Compounds of the formula I, in which L is hydrogen, R³ is n-propyl and X is methyl and the combination of R¹ and R² corresponds for each compound to one row of Table A

### Table 71

Compounds of the formula I, in which L is fluorine, R³ is n-propyl and X is methyl and the combination of R¹ and R² corresponds for each compound to one row of Table A

### Table 72

Compounds of the formula I, in which L is chlorine, R³ is n-propyl and X is methyl and the combination of R¹ and R² corresponds.for each compound to one row of Table A

### Table 73

Compounds of the formula I, in which L is hydrogen, R³ is isopropyl and X is methyl and the combination of R¹ and R² corresponds for each compound to one row of Table A

### Table 74

Compounds of the formula I, in which L is fluorine, R³ is isopropyl and X is methyl and the combination of R¹ and R² corresponds for each compound to one row of Table A

### Table 75

Compounds of the formula I, in which L is chlorine, R³ is isopropyl and X is methyl and the combination of R¹ and R² corresponds for each compound to one row of Table A

### Table 76

Compounds of the formula I, in which L is hydrogen, R³ is 2-fluoroethyl and X is methyl and the combination of R¹ and R² corresponds for each compound to one row of Table A

### Table 77

Compounds of the formula I, in which L is fluorine, R³ is 2-fluoroethyl and X is methyl and the combination of R¹ and R² corresponds for each compound to one row of Table A

### Table 78

Compounds of the formula I, in which L is chlorine, R³ is 2-fluoroethyl and X is methyl and the combination of R¹ and R² corresponds for each compound to one row of Table A

### Table 79

Compounds of the formula I, in which L is hydrogen, R³ is allyl and X is methyl and the combination of R¹ and R² corresponds for each compound to one row of Table A

### Table 80

Compounds of the formula I, in which L is fluorine, R³ is allyl and X is methyl and the combination of R¹ and R² corresponds for each compound to one row of Table A

### Table 81

Compounds of the formula I, in which L is chlorine, R³ is allyl and X is methyl and the combination of R¹ and R² corresponds for each compound to one row of Table A

### Table 82

Compounds of the formula I, in which L is hydrogen, R³ is 2-methoxyethyl and X is methyl and the combination of R¹ and R² corresponds for each compound to one row of Table A

### Table 83

Compounds of the formula I, in which L is fluorine, R³ is 2-methoxyethyl and X is methyl and the combination of R¹ and R² corresponds for each compound to one row of Table A

### Table 84

Compounds of the formula I, in which L is chlorine, R³ is 2-methoxyethyl and X is methyl and the combination of R¹ and R² corresponds for each compound to one row of Table A

TABLE A

| No. | R¹ | R² |
|---|---|---|
| A-1 | H | H |
| A-2 | CH₃ | H |
| A-3 | CH₃ | CH₃ |
| A-4 | CH₂CH₃ | H |
| A-5 | CH₂CH₃ | CH₃ |
| A-6 | CH₂CH₃ | CH₂CH₃ |
| A-7 | CH₂CF₃ | H |
| A-8 | CH₂CF₃ | CH₃ |
| A-9 | CH₂CF₃ | CH₂CH₃ |
| A-10 | CH₂CCl₃ | H |
| A-11 | CH₂CCl₃ | CH₃ |
| A-12 | CH₂CCl₃ | CH₂CH₃ |
| A-13 | CH₂CH₂CH₃ | H |
| A-14 | CH₂CH₂CH₃ | CH₃ |
| A-15 | CH₂CH₂CH₃ | CH₂CH₃ |
| A-16 | CH₂CH₂CH₃ | CH₂CH₂CH₃ |
| A-17 | CH(CH₃)₂ | H |
| A-18 | CH(CH₃)₂ | CH₃ |
| A-19 | CH(CH₃)₂ | CH₂CH₃ |
| A-20 | CH₂CH₂CH₂CH₃ | H |
| A-21 | CH₂CH₂CH₂CH₃ | CH₃ |
| A-22 | CH₂CH₂CH₂CH₃ | CH₂CH₃ |
| A-23 | CH₂CH₂CH₂CH₃ | CH₂CH₂CH₃ |
| A-24 | CH₂CH₂CH₂CH₃ | CH₂CH₂CH₂CH₃ |
| A-25 | (±) CH(CH₃)—CH₂CH₃ | H |
| A-26 | (±) CH(CH₃)—CH₂CH₃ | CH₃ |
| A-27 | (±) CH(CH₃)—CH₂CH₃ | CH₂CH₃ |
| A-28 | (S) CH(CH₃)—CH₂CH₃ | H |
| A-29 | (S) CH(CH₃)—CH₂CH₃ | CH₃ |
| A-30 | (S) CH(CH₃)—CH₂CH₃ | CH₂CH₃ |
| A-31 | (R) CH(CH₃)—CH₂CH₃ | H |
| A-32 | (R) CH(CH₃)—CH₂CH₃ | CH₃ |
| A-33 | (R) CH(CH₃)—CH₂CH₃ | CH₂CH₃ |
| A-34 | (±) CH(CH₃)—CH(CH₃)₂ | H |
| A-35 | (±) CH(CH₃)—CH(CH₃)₂ | CH₃ |
| A-36 | (±) CH(CH₃)—CH(CH₃)₂ | CH₂CH₃ |
| A-37 | (S) CH(CH₃)—CH(CH₃)₂ | H |
| A-38 | (S) CH(CH₃)—CH(CH₃)₂ | CH₃ |
| A-39 | (S) CH(CH₃)—CH(CH₃)₂ | CH₂CH₃ |
| A-40 | (R) CH(CH₃)—CH(CH₃)₂ | H |
| A-41 | (R) CH(CH₃)—CH(CH₃)₂ | CH₃ |
| A-42 | (R) CH(CH₃)—CH(CH₃)₂ | CH₂CH₃ |
| A-43 | (±) CH(CH₃)—C(CH₃)₃ | H |
| A-44 | (±) CH(CH₃)—C(CH₃)₃ | CH₃ |
| A-45 | (±) CH(CH₃)—C(CH₃)₃ | CH₂CH₃ |
| A-46 | (S) CH(CH₃)—C(CH₃)₃ | H |
| A-47 | (S) CH(CH₃)—C(CH₃)₃ | CH₃ |
| A-48 | (S) CH(CH₃)—C(CH₃)₃ | CH₂CH₃ |
| A-49 | (R) CH(CH₃)—C(CH₃)₃ | H |
| A-50 | (R) CH(CH₃)—C(CH₃)₃ | CH₃ |
| A-51 | (R) CH(CH₃)—C(CH₃)₃ | CH₂CH₃ |
| A-52 | (±) CH(CH₃)—CF₃ | H |
| A-53 | (±) CH(CH₃)—CF₃ | CH₃ |
| A-54 | (±) CH(CH₃)—CF₃ | CH₂CH₃ |
| A-55 | (S) CH(CH₃)—CF₃ | H |
| A-56 | (S) CH(CH₃)—CF₃ | CH₃ |
| A-57 | (S) CH(CH₃)—CF₃ | CH₂CH₃ |
| A-58 | (R) CH(CH₃)—CF₃ | H |
| A-59 | (R) CH(CH₃)—CF₃ | CH₃ |
| A-60 | (R) CH(CH₃)—CF₃ | CH₂CH₃ |
| A-61 | (±) CH(CH₃)—CCl₃ | H |
| A-62 | (±) CH(CH₃)—CCl₃ | CH₃ |
| A-63 | (±) CH(CH₃)—CCl₃ | CH₂CH₃ |
| A-64 | (S) CH(CH₃)—CCl₃ | H |
| A-65 | (S) CH(CH₃)—CCl₃ | CH₃ |
| A-66 | (S) CH(CH₃)—CCl₃ | CH₂CH₃ |
| A-67 | (R) CH(CH₃)—CCl₃ | H |
| A-68 | (R) CH(CH₃)—CCl₃ | CH₃ |
| A-69 | (R) CH(CH₃)—CCl₃ | CH₂CH₃ |
| A-70 | CH₂CF₂CF₃ | H |
| A-71 | CH₂CF₂CF₃ | CH₃ |
| A-72 | CH₂CF₂CF₃ | CH₂CH₃ |
| A-73 | CH₂(CF₂)₂CF₃ | H |
| A-74 | CH₂(CF₂)₂CF₃ | CH₃ |
| A-75 | CH₂(CF₂)₂CF₃ | CH₂CH₃ |
| A-76 | CH₂C(CH₃)═CH₂ | H |
| A-77 | CH₂C(CH₃)═CH₂ | CH₃ |
| A-78 | CH₂C(CH₃)═CH₂ | CH₂CH₃ |
| A-79 | CH₂CH═CH₂ | H |
| A-80 | CH₂CH═CH₂ | CH₃ |
| A-81 | CH₂CH═CH₂ | CH₂CH₃ |
| A-82 | CH₂—C≡CH | H |
| A-83 | CH₂—C≡CH | CH₃ |
| A-84 | CH₂—C≡CH | CH₂CH₃ |
| A-85 | cyclopentyl | H |
| A-86 | cyclopentyl | CH₃ |
| A-87 | cyclopentyl | CH₂CH₃ |
| A-88 | cyclohexyl | H |
| A-89 | cyclohexyl | CH₃ |
| A-90 | cyclohexyl | CH₂CH₃ |
| A-91 | CH₂—C₆H₅ | H |
| A-92 | CH₂—C₆H₅ | CH₃ |
| A-93 | CH₂—C₆H₅ | CH₂CH₃ |

| | |
|---|---|
| A-94 | —(CH₂)₂CH═CHCH₂— |
| A-95 | —(CH₂)₂C(CH₃)═CHCH₂— |
| A-96 | —(CH₂)₂CH(CH₃)(CH₂)₂— |
| A-97 | —(CH₂)₃CHFCH₂— |
| A-98 | —(CH₂)₂CHF(CH₂)₂— |
| A-99 | —CH₂CHF(CH₂)₃— |
| A-100 | —(CH₂)₂CH(CF₃)(CH₂)₂— |
| A-101 | —(CH₂)₂O(CH₂)₂— |
| A-102 | —(CH₂)₂S(CH₂)₂— |
| A-103 | —(CH₂)₅— |
| A-104 | —(CH₂)₄— |
| A-105 | —CH₂CH═CHCH₂— |
| A-106 | —CH(CH₃)(CH₂)₃— |
| A-107 | —CH₂CH(CH₃)(CH₂)₂— |
| A-108 | —CH(CH₃)—(CH₂)₂—CH(CH₃)— |
| A-109 | —CH(CH₃)—(CH₂)₄— |
| A-110 | —CH₂—CH(CH₃)—(CH₂)₃— |
| A-111 | —(CH₂)—CH(CH₃)—CH₂—CH(CH₃)—CH₂— |
| A-112 | —CH(CH₂CH₃)—(CH₂)₄— |
| A-113 | —(CH₂)₂—CHOH—(CH₂)₂— |
| A-114 | —(CH₂)—CH═CH—(CH₂)₂— |
| A-115 | —(CH₂)₆— |
| A-116 | —CH(CH₃)—(CH₂)₅— |
| A-117 | —(CH₂)₂—N(CH₃)—(CH₂)₂— |
| A-118 | —N═CH—CH═CH— |
| A-119 | —N═C(CH₃)—CH═C(CH₃)— |
| A-120 | —N═C(CF₃)—CH═C(CF₃)— |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:

Alternaria species on fruit and vegetables,

Bipolaris and Drechslera species on cereals, rice and lawns,

Blumeria graminis (powdery mildew) on cereals,

Botrytis cinerea (gray mold) on strawberries, vegetables, ornamental plants and grapevines,

Erysiphe cichoracearum and Sphaerotheca fuliginea on cucurbits,

Fusarium and Verticillium species on various plants,

Mycosphaerella species on cereals, bananas and peanuts,

Phytophthora infestans on potatoes and tomatoes,

Plasmopara viticola on grapevines,

Podosphaera leucotricha on apples,

Pseudocercosporella herpotrichoides on wheat and barley,

Pseudoperonospora species on hops and cucumbers,

Puccinia species on cereals,

Pyricularia oryzae on rice,

Rhizoctonia species on cotton, rice and lawns,

Septoria tritici and Stagonospora nodorum on wheat,

Uncinula necator on grapevines,

Ustilago species on cereals and sugar cane, and

Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg, preferably 5 to 100 g per 100 kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:

water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,

carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

### 1. Products For Dilution With Water

### A Water-Soluble Concentrates (SL)

10 parts by weight of a compound according to the invention are dissolved in water or in a water-soluble solvent. As an alternative, wetters or other auxiliaries are added. The active compound dissolves upon dilution with water.

### B Dispersible Concentrates (DC)

20 parts by weight of a compound according to the invention are dissolved in cyclohexanone with addition of a dispersant, for example polyvinylpyrrolidone. Dilution with water gives a dispersion.

### C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives.an emulsion.

### D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

### E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.

### F Water-Dispersible Granules And Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

### G Water-Dispersible Powders And Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

### 2. Products To Be Applied Undiluted

### H Dustable Powders (DP)

5 parts by weight of a compound according to the invention are ground finely and mixed intimately with 95% of finely divided kaolin. This gives a dustable product.

### I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

### J ULV Solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:

acylalanines, such as benalaxyl, metalaxyl, ofurace or oxadixyl,

amine derivatives, such as aldimorph, dodine, dodemorph, fenpropimorph, fenpropidin, guazatine, iminoctadine, spiroxamine or tridemorph,

anilinopyrimidines, such as pyrimethanil, mepanipyrim or cyprodinyl,

antibiotics, such as cycloheximide, griseofulvin, kasugamycin, natamycin, polyoxin or streptomycin,

azoles, such as bitertanol, bromoconazole, cyproconazole, difenoconazole, dinitroconazole, enilconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,

dicarboximides, such as iprodione, myclozolin, procymidone or vinclozolin,

dithiocarbamates, such as ferbam, nabam, maneb, mancozeb, metam, metiram, propineb, polycarbamate, thiram, ziram or zineb,

heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,

copper fungicides, such as Bordeaux mixture, copper acetate, copper oxychloride or basic copper sulfate,

nitrophenyl derivatives, such as binapacryl, dinocap, dinobuton or nitrophthalisopropyl,

phenylpyrroles, such as fenpiclonil or fludioxonil,

sulfur,

other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,

strobilurins, such as azoxystrobin, dimoxystrobin, fluoxastrobin, kresoxim-methyl, metominostrobin, orysastrobin, picoxystrobin, pyraclostrobin or trifloxystrobin,

sulfenic acid derivatives, such as captafol, captan, dichlofluanid, folpet or tolylfluanid,

cinnamides and analogous compounds, such as dimethomorph, flumetover or flumorph.

### SYNTHESIS EXAMPLES

With appropriate modification of the starting materials, the procedures given in the synthesis examples below were used to obtain further compounds 1. The compounds obtained in this manner are listed in the table below, together with physical data.

### Example 1

### Preparation of 5-cyano-6-(2,6-difluoro-4-methoxyphenyl)-7-(4-methyl-piperidinyl)-1,2,4-triazolo[1,5a]pyrimidine

A solution of 0.2 g (0.51 mmol) of 5-chloro-6-(2,6-difluoro4-methoxyphenyl)-7-(4-methylpiperidinyl)-1,2,4-triazolo[1,5a]pyrimidine (cf. EP-A 550 113) and 0.4 g (1.5 mmol) of tetrabutylammonium cyanide in 10 ml of acetonitrile was stirred at 20-25° C. for about 14 hours and then at 45° C. for 5 hours. The reaction mixture was then filtered off through silica gel and the filtrate was freed from the solvent. The residue was purified by preparative MPLC on silica gel RP-18 (mobile phase acetonitrile/water). This gave 0.06 g of the title compound as a colorless solid of m.p. 218° C.

¹H-NMR (CDCl₃, δ in ppm): 8.5 (s,1H); 6.55 (d, 2H); 3.9 (s, 3H); 3.8 (d, 2H); 2.9 (t, 2H); 1.7 (d, 2H); 1.6 (m, 1H); 1.4 (m, 2H); 0.95 (d, 3H)

### Example 2

### Preparation of 5-methyl-6-(2,6-difluoro-4-methoxyphenyl)-7-(4-methyl-piperidinyl)-1,2,4-triazolo[1,5a]pyrimidine

A solution of 1 g (2.5 mmol) of 5-chloro-6-(2,6-difluoro4-methoxyphenyl)-7-(4-methylpiperidinyl)-1,2,4-triazolo[1,5a]pyrimidine (cf. EP-A 550 113) and 1 g (6.3 mmol) of sodium dimethylmalonate in 10 ml of acetonitrile was stirred at 70-80° C. for about 3 hours. Another 1 g of sodium dimethylmalonate was then added, and stirring at 70-80° C. was continued for another 3 hours. A yellow precipitate formed. The reaction mixture was the filtered through kieselguhr and the yellow precipitate was taken up in methylene chloride and dilute hydrochloric acid and stirred at 20-25° C. for about 15 min.

After phase separation, the organic phase was dried and freed from the solvent. The residue obtained was taken up in 30 ml of conc. hydrochloric acid, and the reaction mixture was heated under reflux for 5 hours. The reaction mixture was then poured into ice-water and the aqueous phase was extracted with methylene chloride. The combined organic phases were dried and freed from the solvent. This gave 0.45 g of the title compound as a bright solid of m.p. 83° C.

¹H-NMR (CDCl₃, δ in ppm): 8.4 (s,1H); 6.6 (d, 2H); 3.9 (s, 3H); 3.6 (d, 2H); 2.75 (t, 2H); 2.4 (s, 3H); 1.6 (d, 2H); 1.5 (m, 1H); 1.3 (m, 2H); 0.95 (d, 3H)

### Example 3

### Preparation of 5-methoxy-6-(2,6-difluoro-4-methoxyphenyl)-7-(4-methyl-piperidinyl)-1,2,4-triazolo[1,5a]pyrimidine

A solution of 0.2 g (0.51 mmol) of 5-chloro-6-(2,6-difluoro-4-methoxyphenyl)-7-(4-methylpiperidinyl)-1,2,4-triazolo[1,5a]pyrimidine (cf. EP-A 550 113) and 1.5 g of 30% strength sodium methoxide solution in 5 ml of methanol was stirred at 20-25° C. for about 14 hours. The reaction mixture was filtered off through silica gel and freed from the solvent. The residue was purified by preparative MPLC on silica gel RP-18 (mobile phase acetonitrile/water). This gave 0.12 g of the title compound as a yellow resin.

¹H-NMR (CDCl₃, δ in ppm): 8.25 (s, 1H); 6.6 (d, 2H); 4.0 (s, 3H); 3.85 (s, 3H); 3.65 (d, 2H); 2.75 (t, 2H); 1.6 (d, 2H); 1.5 (m,1H); 1.35 (m, 2H); 0.95 (d, 3H)

TABLE I
Compounds of the formula I:

| No. | R¹ | R² | R³ | L | X | Phys. data m.p. [° C.] |
|---|---|---|---|---|---|---|
| I-1 | —(CH₂)₂CH(CH₃)(CH₂)₂— | | CH₃ | F | —CN | 218 |
| I-2 | —(CH₂)₂CH(CH₃)(CH₂)₂— | | CH₃ | F | —CH₃ | 83 |
| I-3 | —(CH₂)₂CH(CH₃)(CH₂)₂— | | CH₃ | F | —OCH₃ | (see ex. 3) |
| I-4 | —CH(CH₃)₂ | H | CH₃ | Cl | —OCH₃ | 169 |

| | | | | | |
|---|---|---|---|---|---|
| I-5 | —(CH₂)₂CH(CH₃)(CH₂)₂— | n-butyl | F | —O-n-butyl | resin |

### Examples For the Action Against Harmful Fungi

The fungicidal action of the compounds of the formula I was demonstrated by the following test:

The active compounds were formulated separately as a stock solution with 0.25% by weight in acetone or DMSO. 1% by weight of the emulsifier Wettol EM 31 (wetting agent having emulsifying and dispersing action based on ethoxylated alkylphenols) was added to this solution. The stock solutions of the active compounds were diluted with water to the stated concentration.

For examples 1 and 2, the active compounds were prepared as a stock solution with 25 mg of active compound which was made up to 10 ml using a mixture of acetone and/or DMSO and the emulsifier Uniperol® EL (wetting agent having emulsifying and dispersing action based on the ethoxylated alkylphenols) in a volume ratio of solvent/emulsifier of 99 to 1. The mixture was then made up with water to 100 ml. This stock solution was diluted with the solvent/emulsifier/water mixture described to the concentration of active compounds stated below.

### Use Example 1

### Activity Against Net Blotch of Barley Caused By Pyrenophora Teres, 1 Day Protective Application

Leaves of potted barley seedlings of the cultivar “Hanna” were sprayed to runoff point with an aqueous suspension having the concentration of active compounds stated below. 24 hours after the spray coating had dried on, the test plants were inoculated with an aqueous spore suspension of Pyrenophora [syn. Drechslera] teres, the net blotch pathogen. The test plants were then placed in a greenhouse at temperatures between 20 and 24° C. and 95 to 100% relative atmospheric humidity. After 6 days, the extent of the development of the disease was determined visually in % infection of the entire leaf area.

In this test, the plants which had been treated with 250 ppm of the compounds I-1, I-2, or I-3 showed an infection of not more than 1%, whereas the untreated plants were 80% infected.

### Use Example 2

### Activity Against Gray Mold On Bell Pepper Leaves Caused By Botrytis Cinerea, Protective Application

Bell pepper seedlings of the cultivar “Neusiedler Ideal Elite” were, after 2 to 3 leaves were well developed, sprayed to runoff point with an aqueous suspension having the concentration of active compounds stated below. The next day, the treated plants were inoculated with a spore suspension of Botrytis cinerea which contained 1.7×10⁶ spores/ml in a 2% aqueous biomalt solution. The test plants were then placed in a dark climatized chamber at 22 to 24° C. and high atmospheric humidity. After 5 days, the extent of the fungal infection on the leaves could be determined visually in %.

In this test, the plants which had been treated with 250 ppm of the compounds I-1, I-2, or I-3 showed no infection, whereas the untreated plants were 80% infected.

### Use Example 3

### Activity Against Alternaria Solani On Tomatoes (Protective)

Leaves of tomato plants of the cultivar “Pixie II” which had been cultivated in pots up to the 4-leaf stage were sprayed to runoff point with an aqueous preparation of active compound which had been prepared from a stock solution of 5% active compound, 94% acetone and 1% emulsifier (Tween 20). After the spray coating had dried on (3-5 hours), the leaves were inoculated with an aqueous spore suspension of Alternaria solani (density 15×10³ spores per ml). The test plants were then placed in climatized chambers at 22 to 24° C. and 96 to 99% relative atmospheric humidity for 36 hours and then cultivated in a greenhouse at 21 to 23° C. and approximately 95% relative atmospheric humidity for a further 2 to 3 days. The extent of the development of the infection on the leaves was then determined visually.

In this test, the plants which had been treated with 250 ppm of the compound I-4 showed no infection, whereas the untreated plants were 100% infected.

1. A 6-(2-fluoro-4-alkoxyphenyl)triazolopyrimidine of the formula I 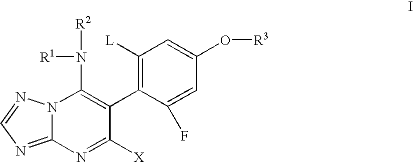
in which the substituents are as defined below:
R¹ C₁-C₈-alkyl, C₁-C₈-haloalkyl, C₃-C₈-cycloalkyl, C₃-C₈-halocycloalkyl, C₂-C₈-alkenyl, C₂-C₈-haloalkenyl, C₃-C₆-cycloalkenyl, C₃-C₆-halocycloalkenyl, C₂-C₈-alkynyl, C₂-C₈-haloalkynyl or phenyl, naphthyl, or a five- or six-membered saturated, partially unsaturated or aromatic heterocycle which contains one to four heteroatoms from the group consisting of O, N and S,
R² is hydrogen or one of the groups mentioned under R¹,
R¹ and R² together with the nitrogen atom to which they are attached may also form a five- or six-membered heterocyclyl or heteroaryl which is attached via N and may contain one to three further heteroatoms from the group consisting of O, N and S as ring member and/or may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, (exo)-C₁-C₆-alkylene and oxy-C₁-C₃-alkyleneoxy;
R³ is C₁-C₈-alkyl, C₁-C₈-haloalkyl, C₃-C₈-alkenyl, C₃-C₈-haloalkenyl, C₃-C₈-alkynyl, C₃-C₈-haloalkynyl, phenyl, phenyl-C₁-C₄-alkyl, mono- or di-(C₁-C₄-alkoxy)-C₁-C₄-alkyl;
R¹, R² and/or R³ may carry one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₈-alkenyl, C₂-C₈-haloalkenyl, C₂-C₆-alkenyloxy, C₂-C₈-alkynyl, C₂-C₈-haloalkynyl, C₃-C₆-alkynyloxy, oxy-C₁-C₃-alkyleneoxy, C₃-C₈-cycloalkenyl, phenyl, naphthyl, a five- or six-membered saturated, partially unsaturated or aromatic heterocycle which contains one to four heteroatoms from the group consisting of O, N and S, where these aliphatic, alicyclic or aromatic groups for their part may be partially or fully halogenated;
L is hydrogen, fluorine or chlorine; and
X is cyano, C₁-C₄-alkyl, C₁-C₄-alkoxy, C₃-C₄-alkenyloxy, C₁-C₂-haloalkoxy or C₃-C₄-haloalkenyloxy.

2. The compound of the formula I as claimed in claim 1 in which X is cyano, C₁-C₄-alkoxy, C₃-C₄-alkenyloxy, C₁-C₂-haloalkoxy or C₃-C₄-haloalkenyloxy.

3. The compound of the formula I as claimed in claim 1 in which X is cyano.

4. The compound of the formula I as claimed in claim 1 in which X is methyl.

5. The compound of the formula I as claimed in claim 1 in which X is methoxy.

6. The compound of the formula I as claimed in claim 1 in which R¹ and R² are as defined below:
R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl, cyclohexyl;
R² is hydrogen or methyl; or
R¹ and R² together form —(CH₂)₂CH(CH₃)(CH₂)₂-, —(CH₂)₂CH(CF₃)(CH₂)₂- or —(CH₂)₂0(CH₂)₂-.

7. A compound of the formula I.1: 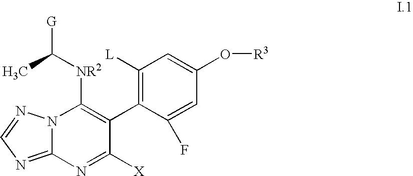
in which
G is C₂-C₆-alkyl, C₁-C₄-alkoxymethyl or C₃-C₆-cycloalkyl;
R² is hydrogen or methyl; and
X is cyano, methyl, methoxy or ethoxy and
L and R³ are as defined in claim 1.

8. A compound of the formula I.2. 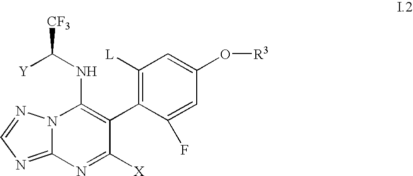
in which Y is hydrogen or C₁-C₄-alkyl and X is cyano, methyl, methoxy or ethoxy and L and R³ are as defined in claim 1.

9. A compound of the formula I.3, 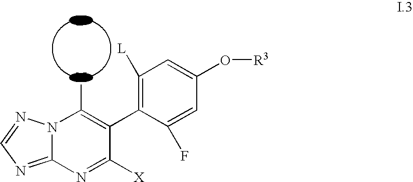
in which
D together with the nitrogen atom forms a five- or six-membered heterocyclyl or heteroaryl which is attached via N and may carry a further heteroatom from the group consisting of O, N and S as ring member and/or may carry one or more substituents from the group consisting of halogen, C₁-C₄-alkyl, CI-C₄-alkoxy and C₁-C₂-haloalkyl;
X is cyano, methyl, methoxy or ethoxy and
L and R³ are as defined in claim 1.

10. The compound of the formula I.3 as claimed in claim 9, in which
L is hydrogen and R³ is methyl.

11. The compound of the formula I, I.1, I.2 and I.3 as claimed in claim 1, in which L is fluorine and R³ is methyl.

12. A process for preparing the compounds of the formula I as claimed in claim 2 which comprises reacting 5-halo-6-(2-halo-4-alkoxyphenyl)triazolopyrimidines of the formula II 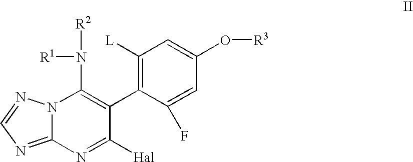
in which Hal is a halogen atom with compounds of the formula III
M—X III
in which M is an ammonium, tetraalkylammonium or alkali metal or alkaline earth metal cation and X is as defined in claim 2.

13. A process for preparing compounds of the formula I as claimed in claim 1 in which X is C₁-C₄-alkyl, by reacting 2-aminotriazole of the formula IV 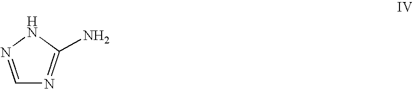
with keto esters of the formula V 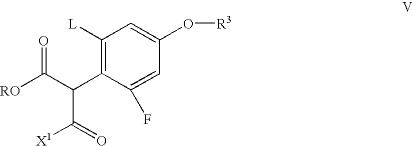
in which R and X¹, independently of one another, are C₁-C₄-alkyl, to give 5-alkyl-7-hydroxy-6-phenyltriazolopyrimidines of the formula VI 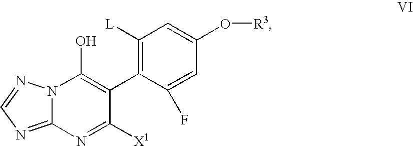
halogenating VI with halogenating agents to give halopyrimidines of the formula VII 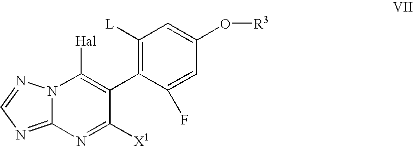
in which Hal is a halogen atom, and reacting VII with amines of the formula VIII 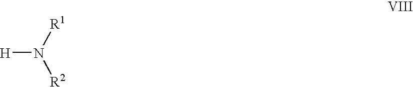
in which R¹ and R² are as defined in formula I.

14. A composition, comprising a solid or liquid carrier and a compound of the formula I as claimed in claim 1.

15. Seed, comprising a compound of the formula I as claimed in claim 1 in an amount of from 1 to 1000 g/100 kg

16. A method for controlling phytopathogenic harmful fungi, which method comprising treating the fungi or the materials, plants, the soil or seed to be protected against fungal attack with an effective amount of a compound of the formula I as claimed in claim 1.

17. The compound of the formula I as claimed in claim 2 in which X is cyano.

18. The compound of the formula I as claimed in claim 2 in which X is methoxy.

19. The compound of the formula I as claimed in claim 2 in which R¹ and R² are as defined below:
R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl, cyclohexyl;
R² is hydrogen or methyl; or
R¹ and R² together form —(CH₂)₂CH(CH₃)(CH₂)₂-, —(CH₂)₂CH(CF₃)(CH₂)₂- or —(CH₂)₂O(CH₂)₂-.

20. The compound of the formula I as claimed in claim 3 in which R¹ and R² are as defined below:
R¹ is CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl, cyclohexyl;
R² is hydrogen or methyl; or
R¹ and R² together form —(CH₂)₂CH(CH₃)(CH₂)₂-, —(CH₂)₂CH(CF₃)(CH₂)₂- or —(CH₂)₂0(CH₂)₂-.
